(19) United States
(12) Patent Application Publication
(10) Pub. No.: US 2012/0071723 A1
(43) Pub. Date: Mar. 22, 2012

(54) ENDOSCOPE APPARATUS AND MEASUREMENT METHOD

(75) Inventors: Takakazu Ishigami, Tokyo (JP); Kiyotomi Ogawa, Tokyo (JP)

(73) Assignee: OLYMPUS CORPORATION, Tokyo (JP)

(21) Appl. No.: 12/887,191

(22) Filed: Sep. 21, 2010

(51) Int. Cl. A61B 1/06; A61B 1/00

(52) U.S. Cl. 600/166; 600/101

(57) ABSTRACT

An endoscope apparatus, which is capable of performing measurement of a subject using a phase-shift method, includes: a main body; an insertion portion connected to the main body; and a plurality of pattern projection units, each of the plurality of pattern projection units including: a pattern window which is provided in a distal end of the insertion portion; and a pattern portion which has a line pattern in which a plurality of lines are periodically disposed with a predetermined period, the lines being parallel to each other, in which: the pattern portions of the plurality of pattern projection units are disposed such that the lines of the line patterns of the pattern portions are parallel to each other, and the line patterns of the pattern portions are shifted from each other by 1/n of 1the predetermined period of the line pattern, where the number of the plurality of pattern projection units assumed to be n (n≧3); and the pattern windows of the plurality of pattern projection units are disposed such that all of the pattern windows have an overlapped portion in a direction perpendicular to the line of the pattern portion.

### BACKGROUND OF THE INVENTION

1. Field of the Invention

The present invention relates to an endoscope apparatus having a function of performing a measurement using a phase-shifting method, and a measurement method through phase-shifting.

2. Description of Related Art

In industrial fields, medical fields, and the like, endoscope apparatuses are used to observe or check inside of a mechanical structure, inside of a patient's body, and the like. There are known endoscope apparatuses which have a function of performing a three-dimensional measurement of a subject using a phase-shifting method (for example, refer to United States Patent Application Publication No. 2009/0225320). The way of the measurement using the phase-shifting method is as follows. A line pattern having a predetermined period is projected on a subject, and an image of the subject is obtained. This procedure is repeated while shifting the phase of the line pattern projected on the subject by a predetermined amount, until the total amount of the phase shift corresponds to the predetermined period of the line pattern. Based on the obtained images, the three-dimensional measurement of the subject is performed using the principle of triangulation.

A conventional endoscope apparatus disclosed in United States Patent Application Publication No. 2009/0225320 includes an observation unit for observing a subject, a pattern projection unit for projecting line patterns on the subject, and a light source which is connected to the pattern projection unit. As shown in FIG. 21, the observation unit includes an observation window 802 provided in a distal surface 801 of an insertion portion of the endoscope apparatus. The pattern projection unit includes a pattern window 803 provided in the distal surface 801, a light guide which connects the light source and the pattern window 803, and a pattern projection portion 804 which is provided on the midway of the light guide and is capable of moving with respect to the light guide. As shown in FIG. 22, the pattern projection portion 804 includes three pattern zones 810a, 810b, and 810c each of which has a line pattern with a predetermined period, and a clear zone 820 on which a line pattern is not formed. The three pattern zones 810a, 810b, and 810c are disposed such that the line patterns of the three pattern zones are shifted from each other by a third of the predetermined period. At the time of the measurement using the phase-shifting method, the pattern zones 810a, 810b, and 810c are sequentially located at the position of the light guide by moving the pattern projection portion 804 with respect to the light guide. As a result, the line patterns of the pattern zones 810a, 810b, and 810c are sequentially projected on the subject, and it is therefore possible to perform the measurement using the phase-shifting method. In addition, when locating the clear zone 820 at the position of the light guide, it is possible to perform a normal observation in which the subject is observed without using the phase-shifting method.

### SUMMARY OF THE INVENTION

An endoscope apparatus according to an aspect of the present invention is capable of performing measurement of a subject using a phase-shift method, and includes: a main body; an insertion portion connected to the main body; and a plurality of pattern projection units, each of the plurality of pattern projection units including: a pattern window which is provided in a distal end of the insertion portion; and a pattern portion which has a line pattern in which a plurality of lines are periodically disposed with a predetermined period, the lines being parallel to each other, in which: the pattern portions of the plurality of pattern projection units are disposed such that the lines of the line patterns of the pattern portions are parallel to each other, and the line patterns of the pattern portions are shifted from each other by 1/n of the predetermined period of the line pattern, where the number of the plurality of pattern projection units assumed to be n (n≧3); and the pattern windows of the plurality of pattern projection units are disposed such that all of the pattern windows have an overlapped portion in a direction perpendicular to the line of the pattern portion.

### BRIEF DESCRIPTION OF THE DRAWINGS

FIG. 1 shows the entire configuration of an endoscope apparatus according to a first embodiment of the invention.

FIG. 2 is a block diagram illustrating the internal configuration of the same endoscope apparatus.

FIG. 3A is a front view of a distal portion of an insertion portion of the same endoscope apparatus, FIG. 3B is a cross-sectional view taken along line A-A in FIG. 3A, and FIG. 3C is a cross-sectional view taken along line B-B in FIG. 3A.

FIG. 4 is a cross-sectional view illustrating the entire configuration of pattern projection units of the same endoscope apparatus.

FIG. 5A is a plan view illustrating an example of a pattern of a pattern portion of the same pattern projection unit, and FIG. 5B is a projection screen of the pattern of FIG. 5A.

FIG. 6 is a cross-sectional view taken along line C-C in FIG. 4.

FIG. 7 is a block diagram illustrating the internal configuration of an endoscope apparatus according to a second embodiment of the invention.

FIG. 8A is a front view of a distal portion of an insertion portion of the same endoscope apparatus, FIG. 8B is a cross-sectional view taken along line A-A in FIG. 8A, and FIG. 8C is a cross-sectional view taken along line B-B in FIG. 8A.

FIG. 9 is a cross-sectional view illustrating the entire configuration of pattern projection units of the same endoscope apparatus.

FIG. 10A is a front view of a distal portion of an insertion portion of an endoscope apparatus according to a third embodiment of the invention, and FIG. 10B is a cross-sectional view taken along line A-A in FIG. 10A.

FIG. 11 is a block diagram illustrating the internal configuration of an endoscope apparatus according to a fourth embodiment of the invention.

FIG. 12A is a front view of a distal portion of an insertion portion of the same endoscope apparatus, FIG. 12B is a cross-sectional view taken along line A-A in FIG. 12A, and FIG. 12C is a cross-sectional view taken along line B-B in FIG. 12A.

FIG. 13 is a cross-sectional view illustrating the entire configuration of pattern projection units of the same endoscope apparatus.

FIG. 14A is a front view of a distal portion of an insertion portion of an endoscope apparatus according to a modification of the fourth embodiment of the invention, and FIG. 14B is a cross-sectional view taken along line A-A in FIG. 14A.

FIG. 15A is a front view of a distal portion of an insertion portion of an endoscope apparatus according to a modification of the first embodiment of the invention,

FIG. 15B is a cross-sectional view taken along line A-A in FIG. 15A, and FIG. 15C is a cross-sectional view taken along line B-B in FIG. 15A.

FIG. 16 is a cross-sectional view illustrating the entire configuration of pattern projection units of the same endoscope apparatus.

FIG. 17A is a front view of a distal portion of an insertion portion of an endoscope apparatus according to another modification of the fourth embodiment of the invention, FIG. 17B is a cross-sectional view taken along line A-A in FIG. 17A, and FIG. 17C is a cross-sectional view taken along line B-B in FIG. 17A.

FIG. 18 is a cross-sectional view illustrating the entire configuration of pattern projection units of the same endoscope apparatus.

FIG. 19 is a cross-sectional view of a proximal portion of an endoscope apparatus according to another modification of the first embodiment of the invention.

FIGS. 20A and 20B are reference views illustrating the arrangement of pattern windows according to another modification of the first embodiment of the invention.

FIG. 21 is a front view of an insertion portion of a conventional endoscope apparatus.

FIG. 22 is a plan view of a pattern projection portion of the conventional endoscope apparatus.

### DETAILED DESCRIPTION OF THE INVENTION

Hereinafter, embodiments of the invention will be described with reference to the drawings.

### First Embodiment

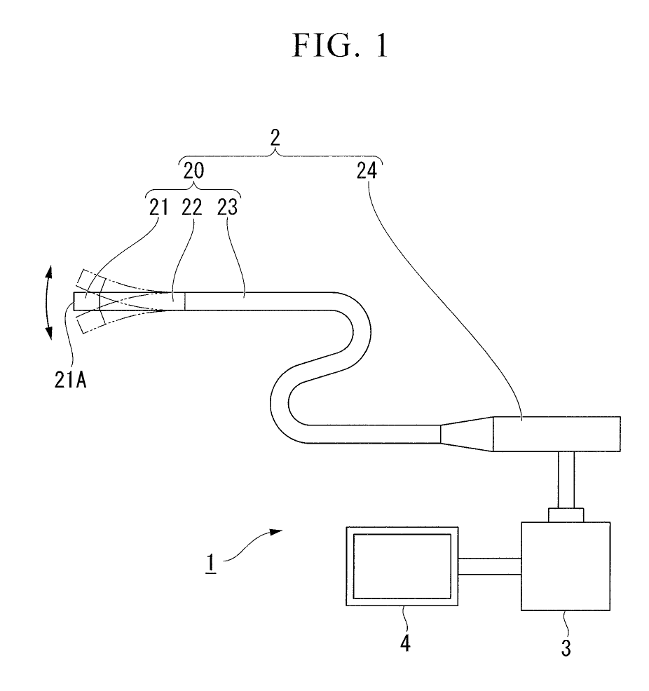

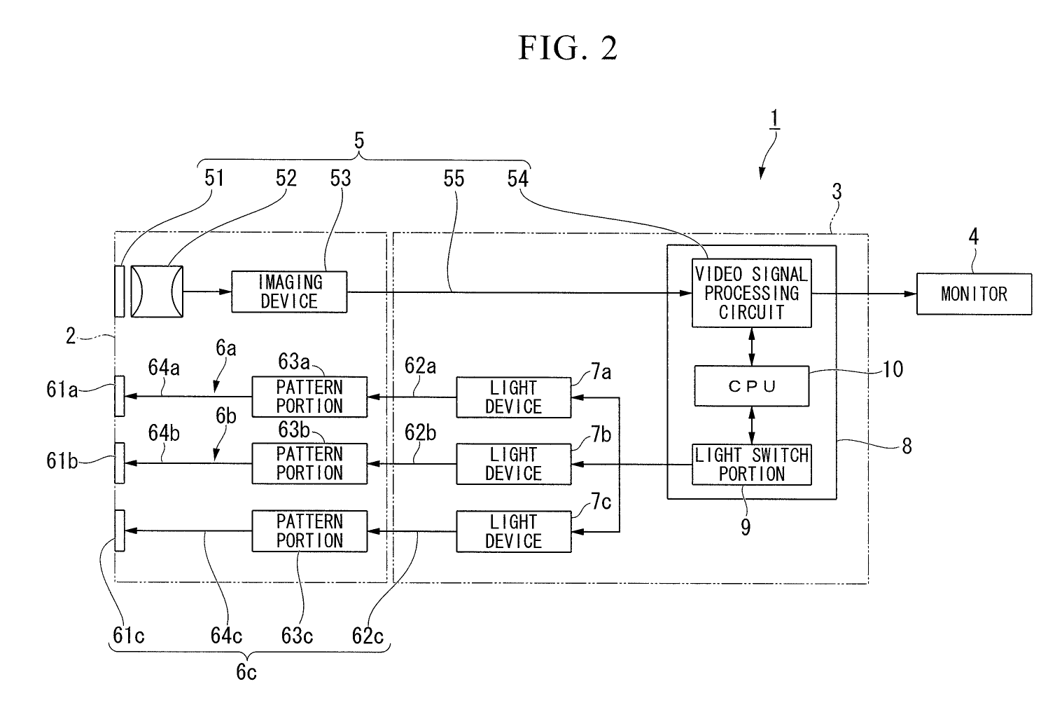

A first embodiment of the invention will be described with reference to FIGS. 1 to 6. FIG. 1 shows the entire configuration of an endoscope apparatus 1 according to the first embodiment of the invention. FIG. 2 is a block diagram illustrating the internal configuration of the endoscope apparatus 1. As shown in FIGS. 1 and 2, the endoscope apparatus 1 includes an endoscope 2, a main body 3 which is connected to the endoscope 2 and has a control portion 8 thereinside, and a monitor 4 which is connected to the main body 3.

An observation unit 5 for observing a subject and a plurality (three in the present embodiment) of pattern projection units 6a, 6b, and 6c are provided in the endoscope 2 and the main body 3. The plurality of pattern projection units 6a, 6b, and 6c projects on the subject patterns for measurement using the phase-shifting method. A plurality (three in the present embodiment) of light devices 7a, 7b, and 7c is provided in the main body 3. Known illuminations such as halogen lamps and LEDs may be employed as the light devices 7a, 7b, and 7c.

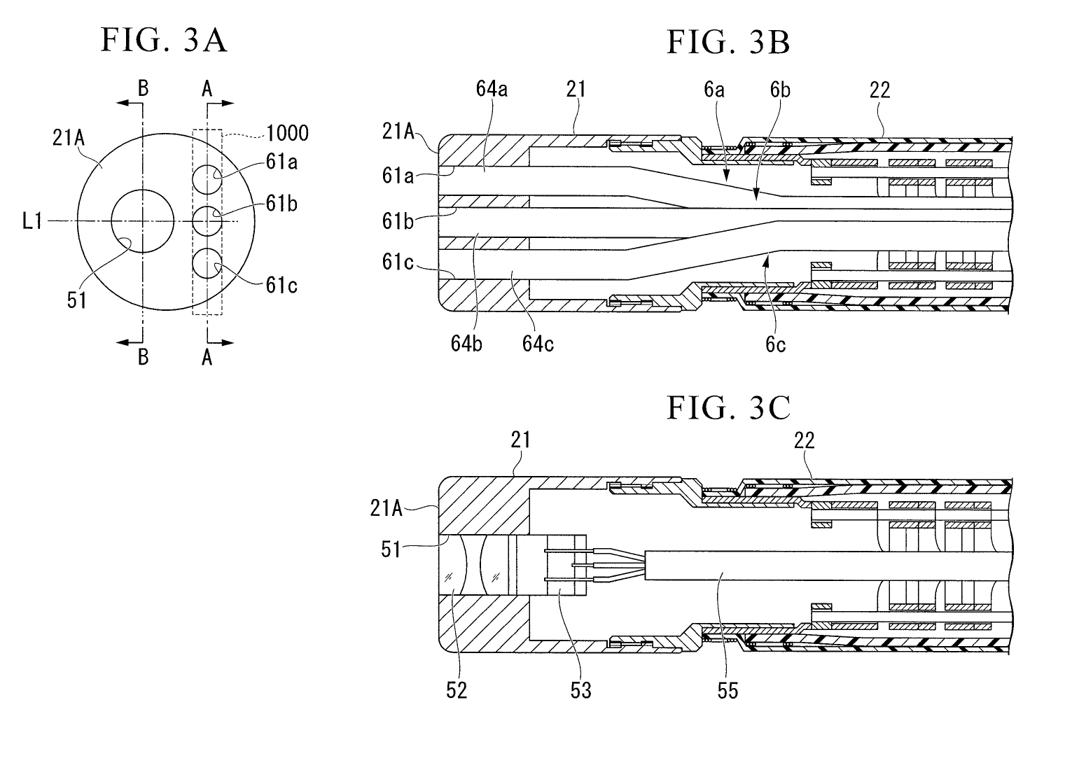

The endoscope 2 has a long and thin insertion portion 20, and an operation portion 24 which performs an operation required in executing various kinds of operation controls of the entire apparatus. The insertion portion 20 includes a hard distal portion 21 which is formed of a cylindrical shape having a distal surface 21A, a bent portion 22 capable of being bent, for example, in the vertical and horizontal directions, and a flexible tube portion 23 with the flexibility, sequentially from the distal side. As shown in FIG. 3A, an observation window 51 (described later) of the observation unit 5 and pattern windows 61a, 61b, and 61c (described later) of the plurality of pattern projection units 6a, 6b, and 6c are provided in the distal surface 21A.

A video signal processing circuit 54 (described later), a light switch portion 9 which switches the emission of light by controlling on-off of the plurality of light devices 7a, 7b, and 7c, and a CPU 10 which performs an operation control of these portions are provided in the control portion 8 of the main body 3.

The configuration of the observation portion 5 will be described. FIG. 3C is a cross-sectional view of the distal portion 21 taken along line B-B in FIG. 3A. As shown in FIGS. 2 and 3C, the observation unit 5 is configured of: the observation window 51 which is provided in the distal surface 21A and has a circular cross-section along the distal surface 21A; an objective optical system 52 which is provided in the distal portion 21; an imaging device 53 such as a CCD (Change Coupled Device); the video signal processing circuit 54 which is built in the control portion 8 of the main body 3; and a signal cable 55 which connects the imaging device 53 and the video signal processing circuit 54. The imaging device 53 is disposed at the image location of the objective optical system 52.

A subject image is formed through the objective optical adaptor 52, and is photoelectrically converted into an image signal by the imaging device 53. The image signal is input from the imaging device 53 to the video signal processing circuit 54 through the signal cable 55, and is converted into a video signal (image data) in the video signal processing circuit 54. The subject image is displayed on the monitor 4 based on the video signal.

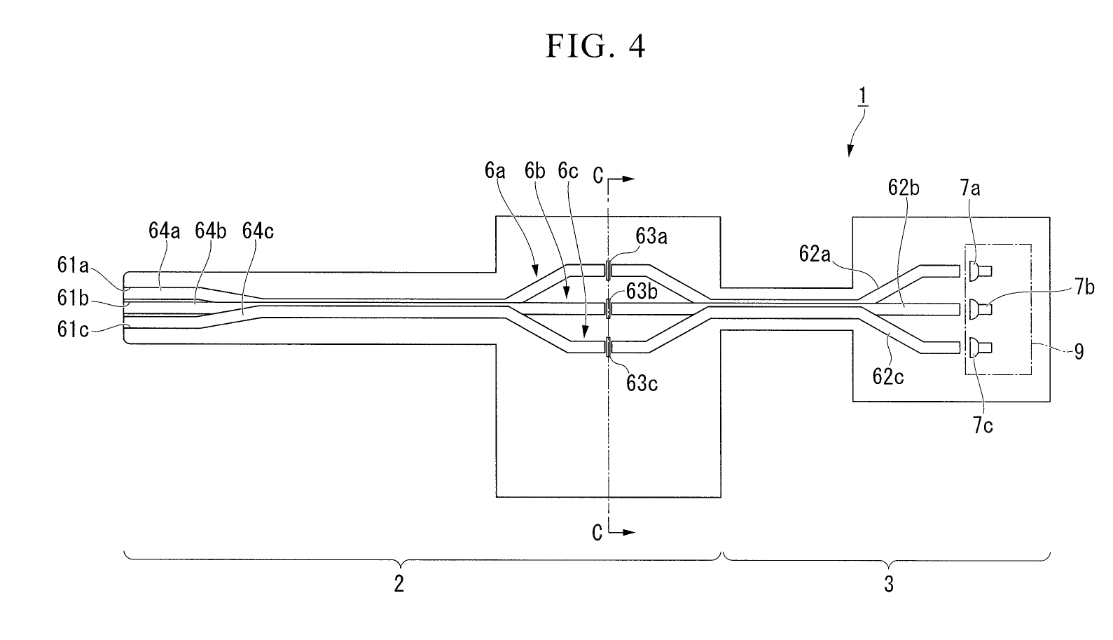

Next, the configuration of the pattern projection units 6a, 6b, and 6c will be described. FIG. 3B is a cross-sectional view of the distal portion 21 taken along line A-A in FIG. 3A. FIG. 4 is a cross-sectional view of the endoscope apparatus 1, and shows the entire configuration of the pattern projection units 6a, 6b, and 6c. As shown in FIGS. 2, 3B and 4, the pattern projection unit 6a includes: the pattern window 61a which is provided in the distal surface 21A and has a circular cross-section along the distal surface 21A; an emitting portion 62a; a pattern portion 63a; and a coherent fiber 64a such as an image guide fiber. Note that the pattern projection units 6b and 6c have the same configuration as that of the pattern projection unit 6a except of the arrangement of a line pattern of the pattern portion described later. Therefore, regarding the pattern projection units 6b and 6c, the same parts as those of the pattern projection unit 6a are designated with the same reference numerals but the trailing alphabets thereof are designated with “b” and “c” instead of “a”, respectively, and an explanation thereof will be omitted. The same holds for the following embodiments.

The emitting portion 62a is provided in the main body 3, and emits light to the outside via the pattern window 61a. In this embodiment, the emitting portion 62a is a light guide which connects the light device 7a and the pattern portion 63a.

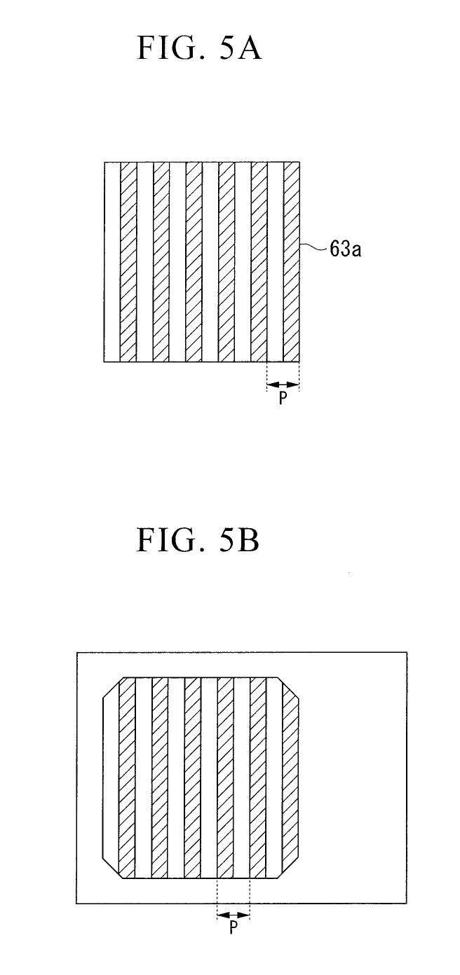

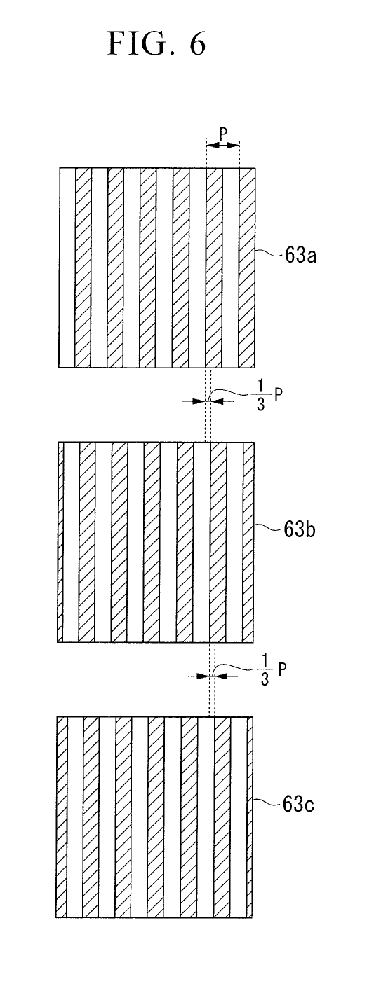

The pattern portion 63a is provided in the insertion portion 20 between the pattern window 61a and the emitting portion 62a. A pattern of the pattern portion 63a is shown in FIG. 5A, and a projection screen of the pattern is shown in FIG. 5B. The pattern portion 63a has a line pattern in which a plurality of lines parallel to each other is periodically disposed with a predetermined period P. As shown in FIG. 6, similar to the pattern portion 63a, the pattern portions 63b and 63c have line patterns in which the plurality of lines parallel to each other is periodically disposed with the predetermined period P. The pattern portions 63a, 63b, and 63c are disposed such that the line patterns thereof are shifted from each other by a third of the predetermined period P. In other words, the pattern portions 63a, 63b, and 63c are disposed such that the phases of the line patterns thereof are shifted from each other by 2π/3.

The coherent fiber 64a is provided in the insertion portion 20, and connects the pattern window 61a and the pattern portion 63a.

As shown in FIG. 3A, the pattern windows 61a, 61b, and 61c have the same shape, and are disposed such that the line A-A connecting the centers of the pattern windows 61a, 61b, and 61c to each other is parallel to the lines of the pattern portions 63a, 63b, and 63c. A dotted line shows an overlapped portion 1000 in which all of the pattern windows 61a, 61b, and 61c overlap in the direction perpendicular to the line of the pattern portion. Further, the observation window 51 is disposed such that a line L1 connecting the center of the observation window 51 and the center of the middle pattern window 61b to each other is perpendicular to the line A-A (i.e., the line of the pattern portion) connecting the centers of the pattern windows 61a, 61b, and 61c.

The light devices 7a, 7b, and 7c and the light switch portion 9 will be described. The light device 7a is connected to the pattern projection unit 6a. Light from the light device 7a passes through the emitting portion 62a, the pattern portion 63a, the coherent fiber 64a and the pattern window 61a, and then is emitted to the outside. As a result, in the pattern portion 63a, the line pattern (first pattern) of the pattern portion 63a is formed on the light from the light device 7a. Similarly, the light device 7b is connected to the pattern projection unit 6b, and the line pattern (second pattern) of the pattern portion 63b is formed on the light from the light device 7b. The light device 7c is connected to the pattern projection unit 6c, and the line pattern (third pattern) of the pattern portion 63c is formed on the light from the light device 7c. The plurality of pattern projection units 6a, 6b, and 6c are arranged such that the line A-A connecting the centers of the emitting lights on the distal surface 21A (i.e., on the pattern windows 61a, 61b, and 61c) is parallel to the lines of the pattern portions 63a, 63b, and 63c. With this arrangement, the first, second, and third patterns projected on the subject via the pattern portions 63a, 63b, and 63c are shifted from each other by exactly a third of the predetermined period P. Therefore, it is possible to perform measurement using the phase-shifting method with accuracy.

As shown in FIGS. 2 and 4, the light devices 7a, 7b, and 7c are connected to the light switch portion 9. The emission of light is switched by the light switch portion 9 controlling on-off of the light devices 7a, 7b, 7c in accordance with the control of the CPU 10.

Next, the measurement procedure using the phase-shifting method by the endoscope apparatus 1 will be described.

First, in accordance with the control of the CPU 10, the light switch portion 9 turns on only one (for example, the light device 7a) of the plurality of the light devices, and turns off the other light devices (for example, the light devices 7b and 7c). As a result, since light is emitted only from the pattern projection unit 6a which is connected to the on-state light device 7a, the line pattern (the first pattern) of the pattern portion 63a is projected on the subject. Then, a first subject image, on which the line pattern of the pattern portion 63a is projected, is imaged (First step). Subsequently, the light switch portion 9 turns on only the light device 7b. As a result, the line pattern (the second pattern) of the pattern portion 63b is projected on the subject, and a second subject image, on which the line pattern of the pattern portion 63b is projected, is imaged (Second step). Subsequently, the light switch portion 9 turns on only the light device 7c. As a result, the line pattern (the third pattern) of the pattern portion 63c is projected on the subject, and a third subject image, on which the line pattern of the pattern portion 63c is projected, is imaged (Third step). With this procedure, it is possible to obtain three subject images (i.e., the first, second and third subject images) on which the line patterns which are shifted from each other by a third of the period P are projected. Based on the first, second and third images, a three-dimensional shape of the subject is measured using the principle of triangulation.

Here, at the time of a normal observation in which the subject is observed through the observation window 51 without using the phase-shifting method, the light switch portion 9 turns on all the light devices 7a, 7b, and 7c in accordance with the control of the CPU 10. As a result, since light is emitted from all the pattern projection units 6a, 6b, and 6c, it is possible to perform the normal observation of the subject in a state where light whose pattern almost disappears is projected on the subject.

In the endoscope apparatus 1 of the present embodiment, the light devices 7a, 7b, and 7c which are connected to the pattern projection units 6a, 6b, and 6c, respectively, are provided, and on-off of the light devices 7a, 7b, and 7c is controlled by the light switch portion 9. As a result, only by switching the emission of light with the light switch portion 9, it is possible to subsequently project on the subject the line patterns of the pattern portions 63a, 63b, and 63c of the pattern projection units 6a, 6b, and 6c to perform measurement using the phase-shifting method. Therefore, since there is no need to additionally provide a mechanism for moving a pattern projection unit or a light device, it is possible to reduce the size and the cost of the endoscope apparatus. In addition, since measurement using the phase-shifting method is performed only by controlling on-off of the light devices 7a, 7b, and 7c with the light switch portion 9, the endoscope apparatus 1 of the present embodiment is reliable even when it is used for a long time. In addition, since the positions of the pattern projection units 6a, 6b, and 6c and the light devices 7a, 7b, and 7c are fixed, the projection position of each of the line patterns of pattern portions 63a, 63b, and 63c on the subject is not misaligned. Therefore, it is possible to perform measurement using the phase-shifting method with accuracy. In addition, by turning on all the light devices 7a, 7b, and 7c such that light is emitted from all the pattern projection units 6a, 6b, and 6c, light whose pattern almost disappears is projected on the subject, and the normal observation of the subject can be performed under this light. Therefore, since there is no need to additionally provide an illumination unit for the normal observation, it is possible to further reduce the size of the endoscope apparatus.

### Second Embodiment

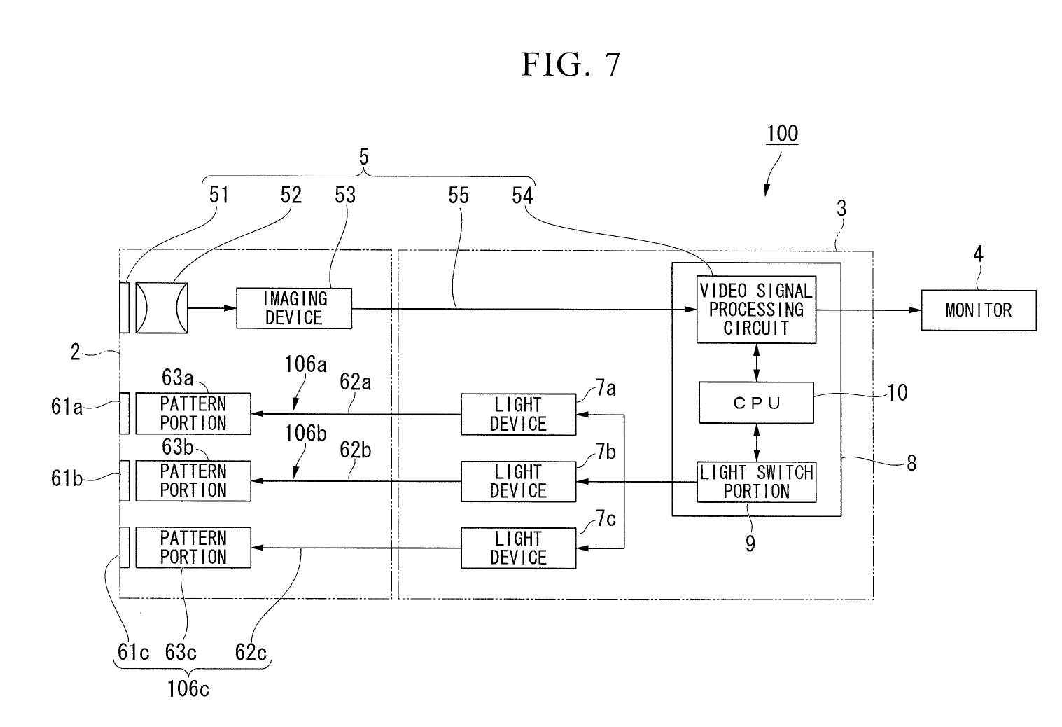

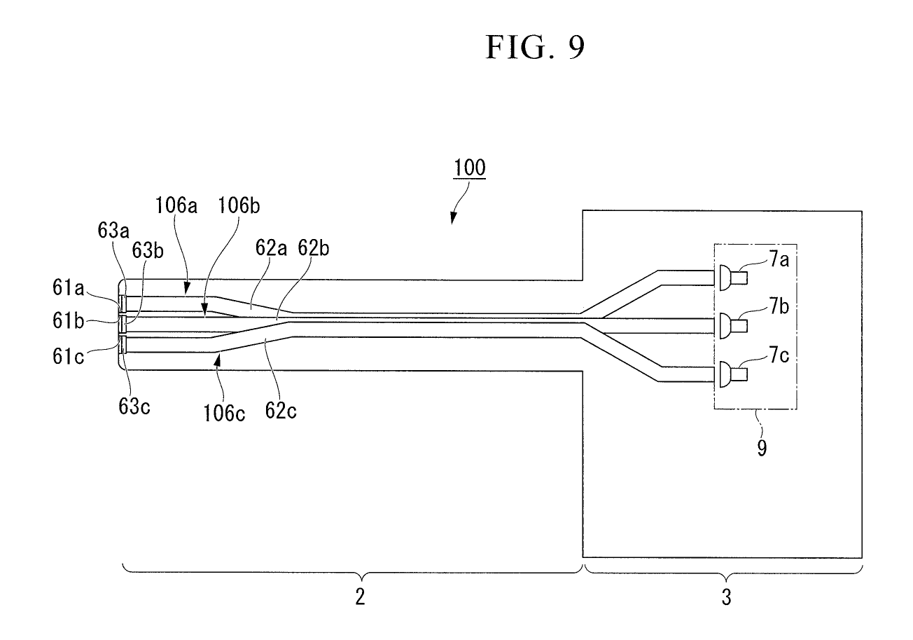

A second embodiment of the invention will be described with reference to FIGS. 7 to 9. An endoscope apparatus 100 of the second embodiment is different from the endoscope apparatus 1 of the first embodiment in that a pattern portion of a pattern projection unit is provided in the distal portion 21 of the insertion portion 20 and a coherent fiber connecting a pattern window and a pattern portion is not provided. Hereinafter, the common elements to those of the above-described embodiment(s) are designated with the same reference numerals and an explanation thereof will be omitted.

FIG. 7 is a block diagram illustrating the internal configuration of the endoscope apparatus 100. A pattern projection unit 106a of the present embodiment is configured of a pattern window 61a, an emitting portion 62a, and a pattern portion 63a.

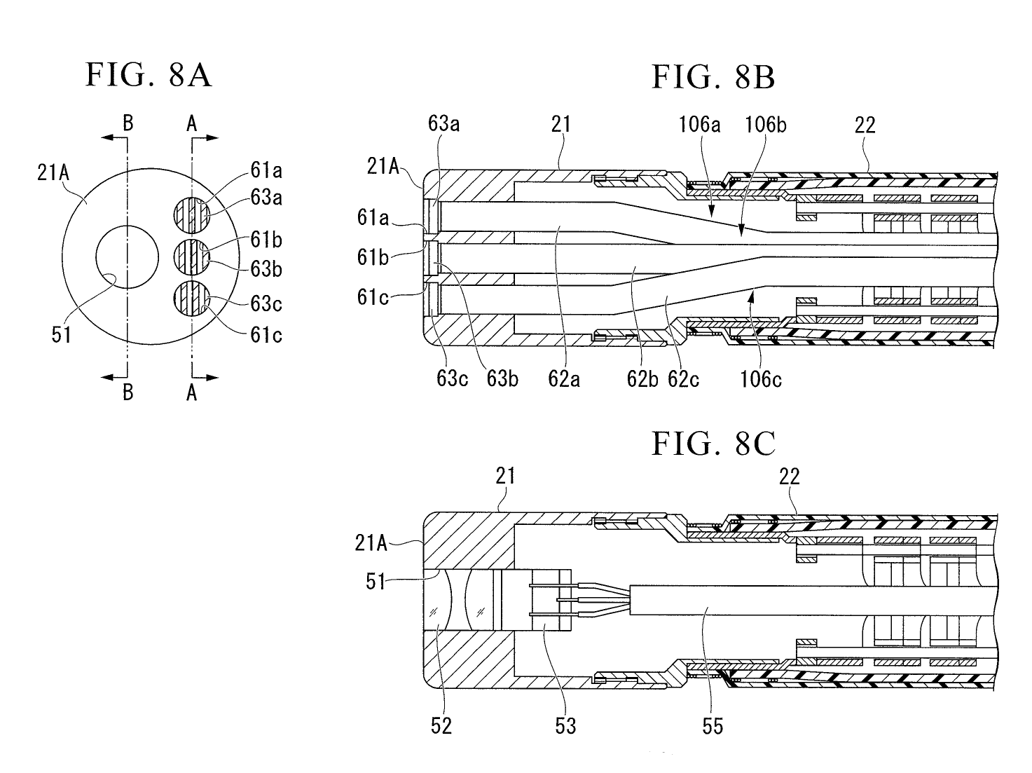

As shown in FIGS. 8A and 8B, the pattern portion 63a is provided in the distal portion 21 of the insertion portion 20 immediately behind the pattern window 61a. As a result, the pattern portion 63a is exposed to the outside via the pattern window 61a. Note that the pattern shape and the arrangement of the pattern portions 61a, 63b, and 63c are the same as those in the first embodiment.

The emitting portion 62a is a light guide which connects the light device 7a and the pattern portion 63a.

Light from the light device 7a passes through the emitting portion 62a, and is emitted to the outside via the pattern portion 63a and the pattern window 61a which are provided in the distal portion 21.

According to the endoscope apparatus 100 of the present embodiment, similar to the endoscope apparatus 1 of the first embodiment, only by switching the emission of light with the light switch portion 9, it is possible to subsequently project on the subject the line patterns of the pattern portions 63a, 63b, and 63c of the pattern projection units 106a, 106b, and 106c to perform measurement using the phase-shifting method. Further, the pattern portions 63a, 63b and 63c are provided in the distal portion 21 of the insertion portion 20 and light from the pattern portions 63a, 63b and 63c are directly emitted to the outside via the pattern windows 61a, 61b and 61c, respectively. Therefore, since a coherent fiber which connects the pattern portion and the pattern window is unnecessary in the present embodiment, it is possible to further reduce the cost of the endoscope apparatus.

### Third Embodiment

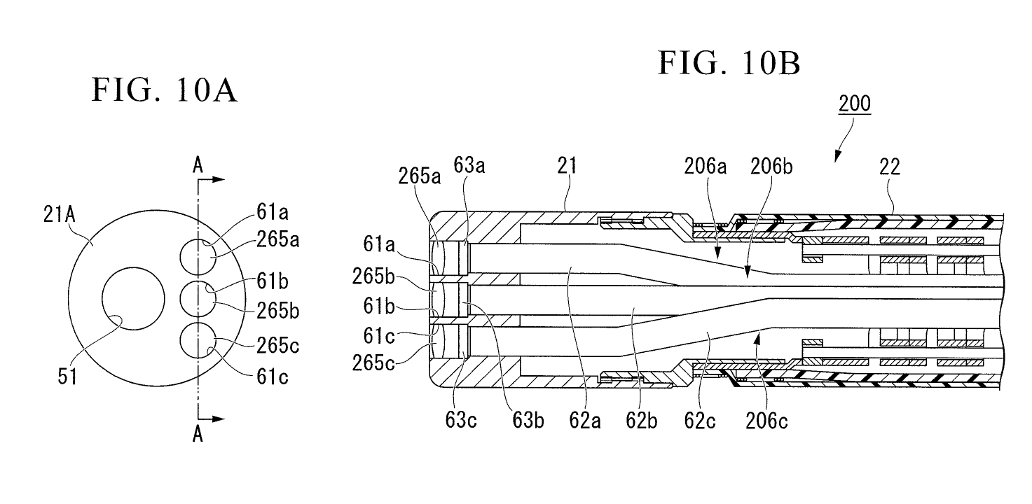

A third embodiment of the invention will be described with reference to FIGS. 10A and 10B. An endoscope apparatus 200 of the third embodiment is different from the endoscope apparatus 100 of the second embodiment in that a lens (projection optical system) is provided between a pattern window and a pattern portion.

As shown in FIG. 10B, a pattern projection unit 206a of the present embodiment includes a pattern window 61a, emitting portion 62a, a pattern portion 63a, and a lens 265a which is provided between the pattern window 61a and the pattern portion 63a.

Light from the light device 7a passes through the emitting portion 62a, and is emitted to the outside via the pattern portion 63a, the lens 265a, and the pattern window 61a. Since the lens 265a can change the focal length of light from the light device 7a to a value appropriate for imaging or observation of the subject, it is possible to perform the observation more clearly.

According to the endoscope apparatus 200 of the present embodiment, similar to the endoscope apparatuses 1 and 100 of the first and second embodiments, only by switching the emission of light with the light switch portion 9, it is possible to subsequently project on the subject the line patterns of the pattern portions 63a, 63b, and 63c of the pattern projection units 206a, 206b, and 206c to perform measurement using the phase-shifting method. In addition, with the lenses 265a, 265b, and 265c, it is possible to set the focal length of light emitted from the pattern portions 63a, 63b, and 63c to any value in accordance with the distance between the endoscope apparatus 200 and the subject. Therefore, it is possible to clear up the line patterns projected on the subject. Particularly, when the focal length of the lenses 265a, 265b, and 265c is set in accordance with the focal length of the objective optical system 52 of the observation unit 5, it is possible to perform the observation more clearly.

### Fourth Embodiment

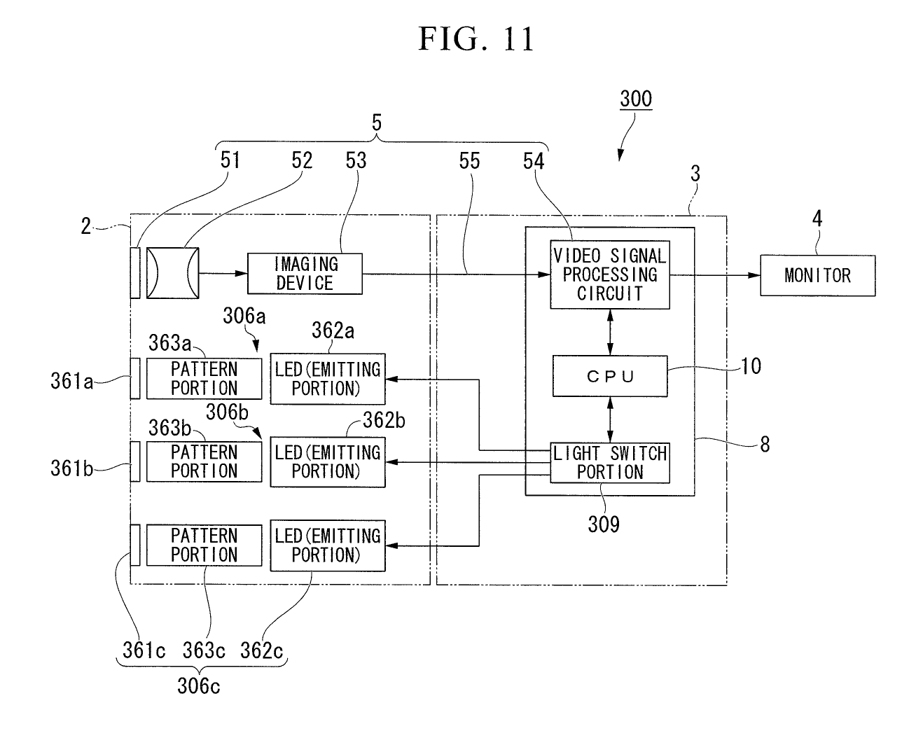

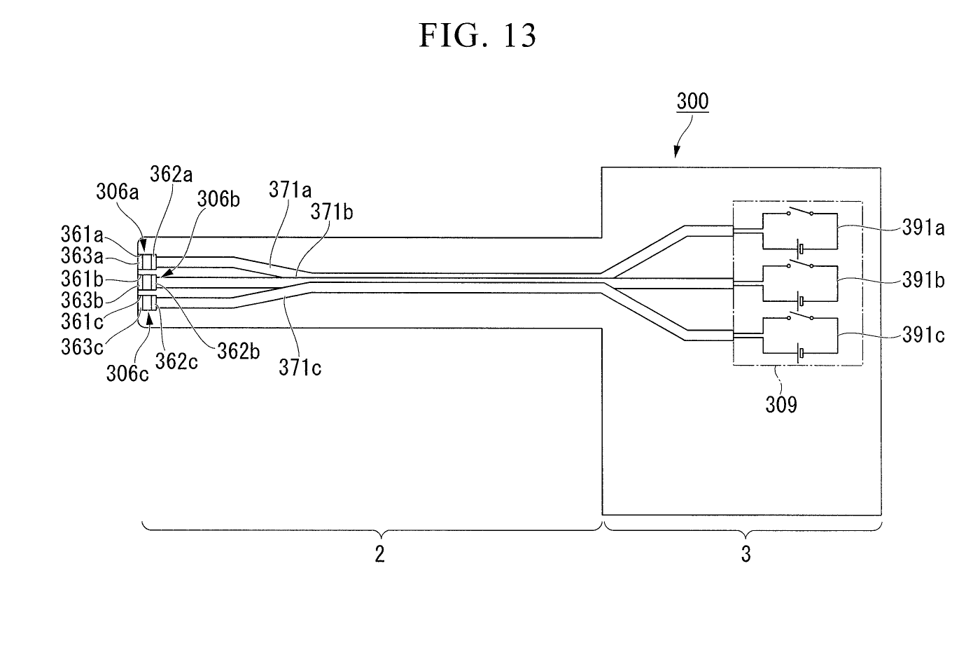

Next, a fourth embodiment of the invention will be described with reference to FIGS. 11 to 13. An endoscope apparatus 300 of the fourth embodiment is different from the endoscope apparatus 1 of the first embodiment in that a light device is not provided in the main body 3, and instead of the light device, an LED (emission member) as an emitting portion is provided in the distal end of the insertion portion 20.

FIG. 11 is a block diagram illustrating the internal configuration of the endoscope apparatus 300. A pattern projection unit 306a of the present embodiment is configured of a pattern window 361a, an emitting portion 362a made of an emission member such as an LED, and a pattern portion 363a.

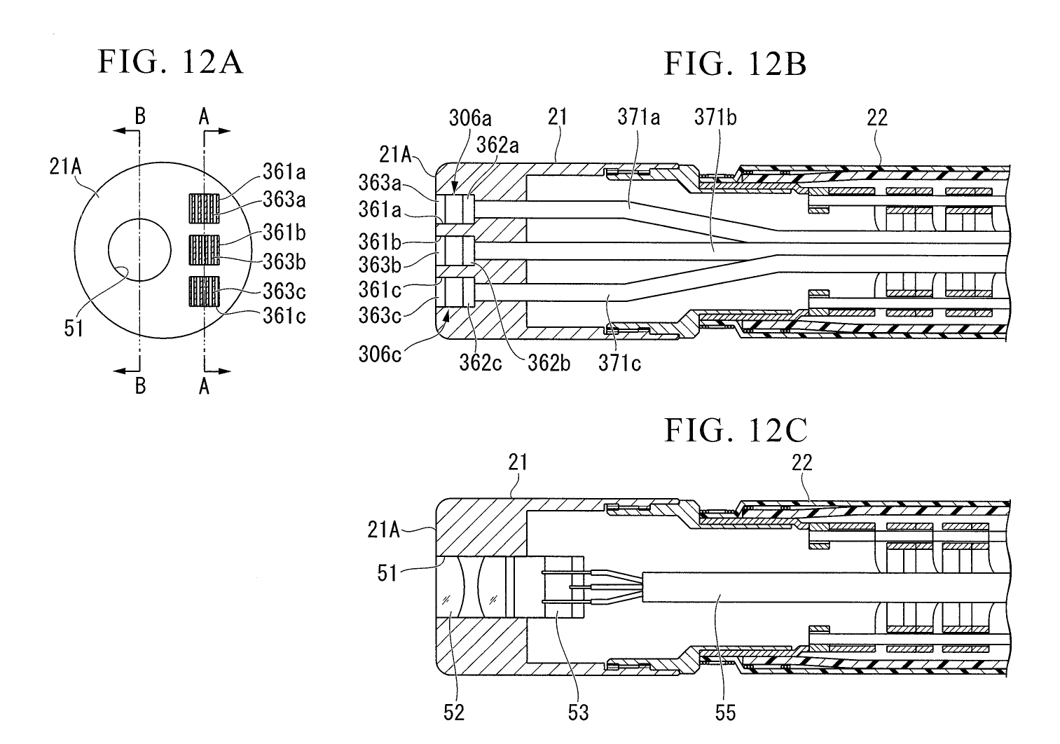

As shown in FIGS. 12A and 12B, the cross-section of the pattern window 361a along the distal surface 21A is a rectangular shape corresponding to the shape of the emitting portion 362a. Further, the pattern portion 363a is provided in the distal portion 21 of the insertion portion 20 immediately behind the pattern window 361a. As a result, the pattern portion 363a is exposed to the outside via the pattern window 361a. Similar to the first embodiment, each of the pattern portions 363a, 363b, and 363c of the pattern projection units 306a, 306b, and 306c includes a line pattern in which a plurality of lines parallel to each other is periodically disposed with a predetermined period P. The pattern portions 363a, 363b, and 363c are disposed such that the line patterns are shifted from each other by a third of the predetermined period P.

The emitting portion 362a made of the emission member is provided in the distal portion 21 of the insertion portion 20 immediately behind the pattern portion 363a. The emitting portions 362a, 362b, and 362c are connected to power sources 391a, 391b , and 391c of a light switch portion 309 via power cables 371a, 371b, and 371c, respectively. The light switch portion 309 independently controls on-off of the power source 391a, 391b, and 391c in accordance with the control of the CPU 10. Thereby, the light switch portion 309 switches the emission of light between the emitting portions 362a, 362b, and 362c.

In the endoscope apparatus 300 of the present embodiment, the emitting portions 362a, 362b, and 362c as emission members are provided in the pattern projection units 306a, 306b, and 306c, respectively, and the light switch portion 309 controls on-off of the emitting portions 362a, 362b, and 362c. As a result, only by switching the emission of light with the light switch portion 309, it is possible to subsequently project on the subject the line patterns of the pattern portions 363a, 363b, and 363c of the pattern projection units 306a, 306b, and 306c to perform measurement using the phase-shifting method. In addition, since the LEDs (emission members) are used as the emitting portions 362a, 362b, and 362c, there is no need to provide a light device in the main body 3. Accordingly, it is possible to further reduce the size of the endoscope apparatus.

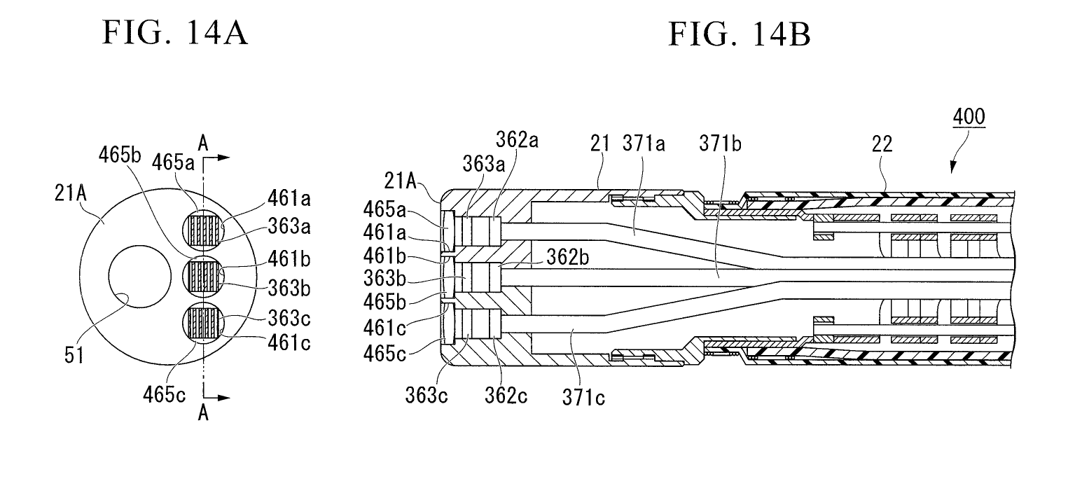

Note that as a modification shown in FIGS. 14A and 14B, lenses (projection optical systems) 465a, 465b, and 465c may be provided between the pattern windows and the pattern portions, respectively. In this case, the cross-section of pattern windows 461a, 461b, and 461c is a circular shape corresponding to the shape of the lenses 465a, 465b, and 465c.

According to an endoscope apparatus 400 of this modification, with the lenses 465a, 465b, and 465c, it is possible to set the focal length of light emitted from the pattern portions 363a, 363b, and 363c to any value in accordance with the distance between the endoscope apparatus 400 and the subject. Therefore, it is possible to clear up the line patterns projected on the subject. Further, when the focal length of the lenses 465a, 465b, and 465c is set in accordance with the focal length of the objective optical system 52 of the observation unit 5, it is possible to perform the observation more clearly.

While preferred embodiments of the invention have been described and illustrated above, it should be understood that these are exemplary of the invention and are not to be considered as limiting. Additions, omissions, substitutions, and other modifications can be made without departing from the scope of the present invention. Accordingly, the invention is not to be considered as being limited by the foregoing description, and is only limited by the scope of the appended claims.

For example, in the above-described embodiments, the normal observation is performed by emitting light from all the pattern projection units. However, a unit for illuminating the subject at the time of the normal observation may be additionally provided.

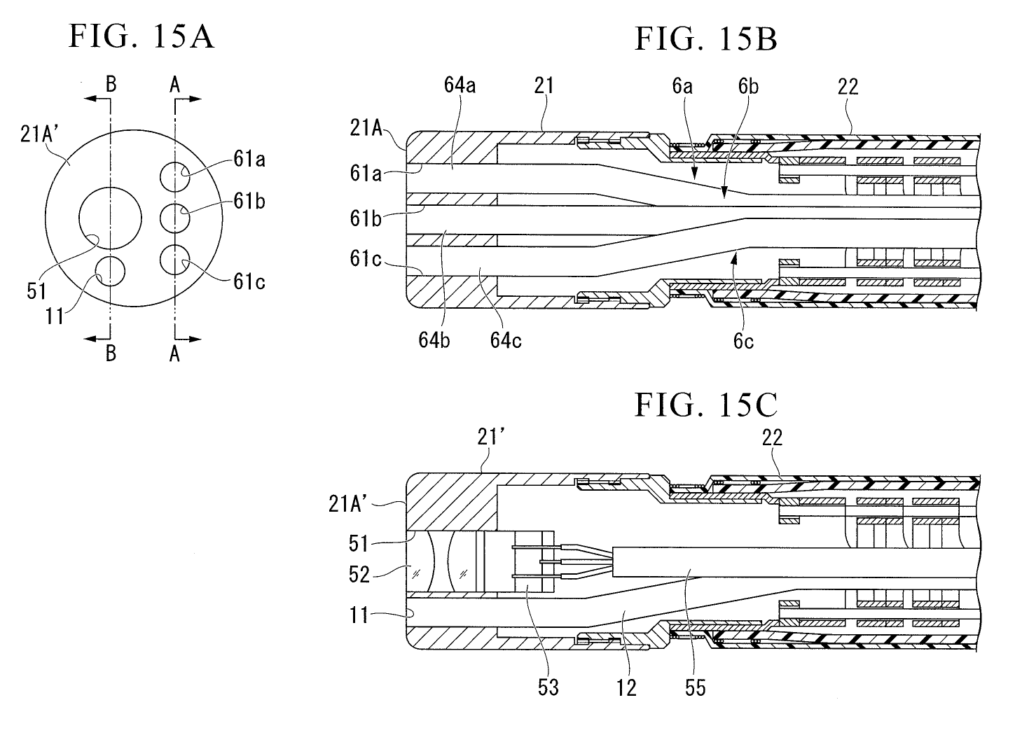

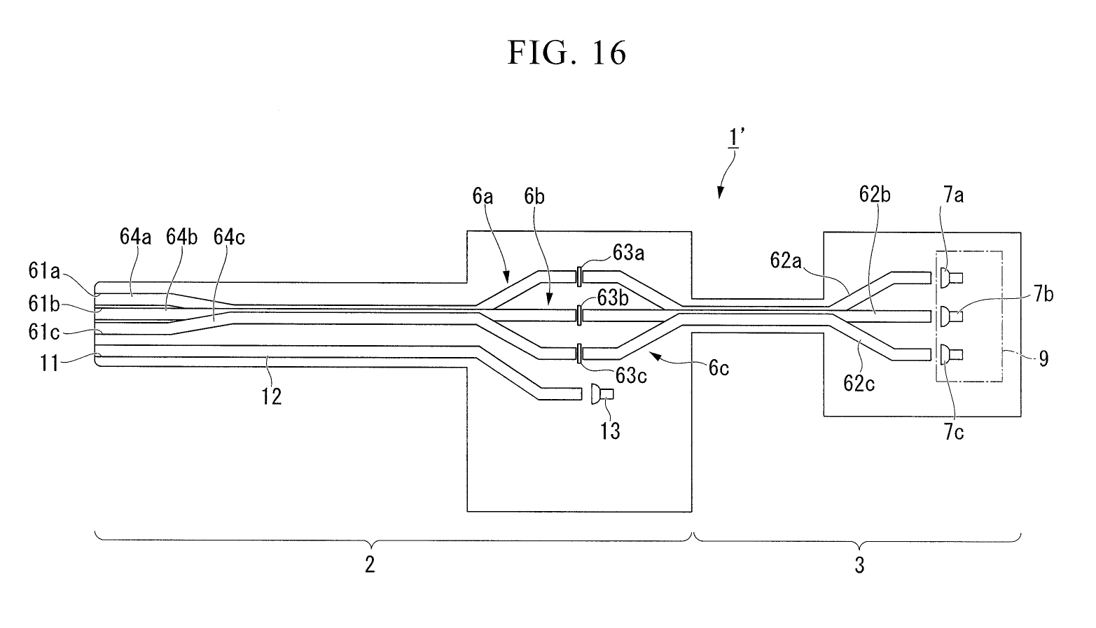

Specifically, as a modification of the first embodiment shown in FIGS. 15A to 16, an endoscope apparatus 1′ may further include a normal illumination window 11 which is provided in a distal surface 21A′ of a distal portion 21′, a light device 13 for the normal observation, and a light guide 12 which connects the normal illumination window 11 and the light device 13. At the time of the normal observation, the light switch portion 9 turns off the light devices 7a, 7b, and 7c, and turns on the light device 13, in accordance with the control of the CPU 10. As a result, since light only from the on-state light device 13 is projected on the subject, it is possible to observe the subject under light suitable for the normal observation.

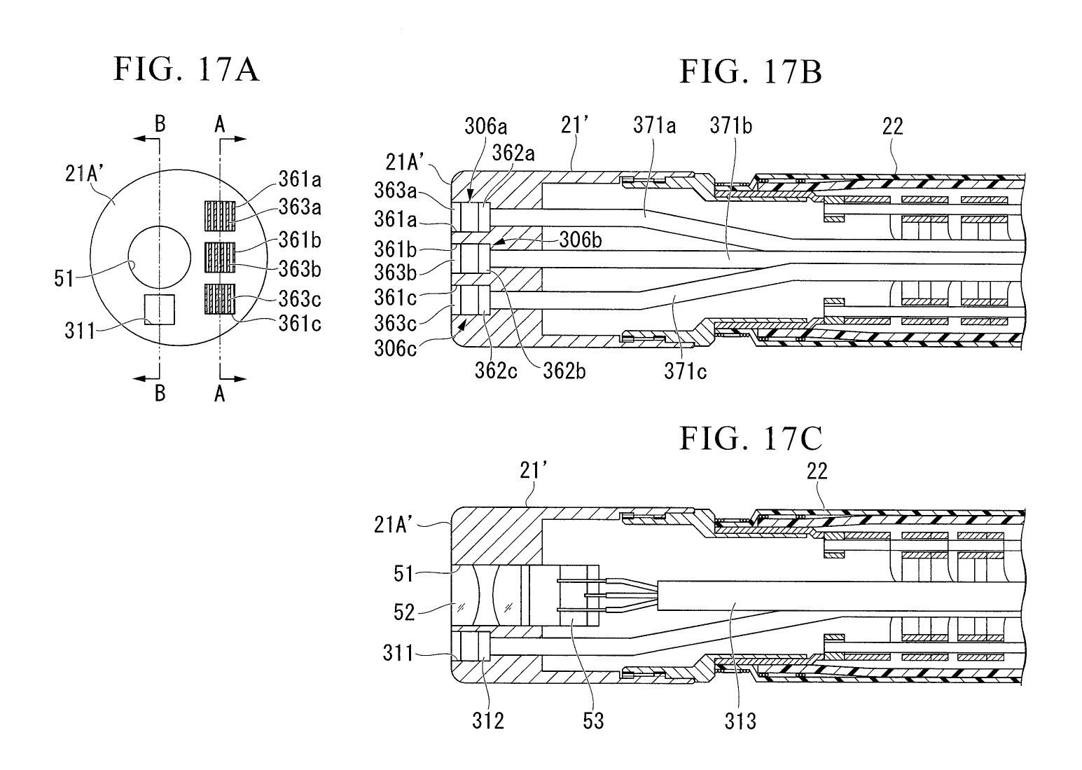

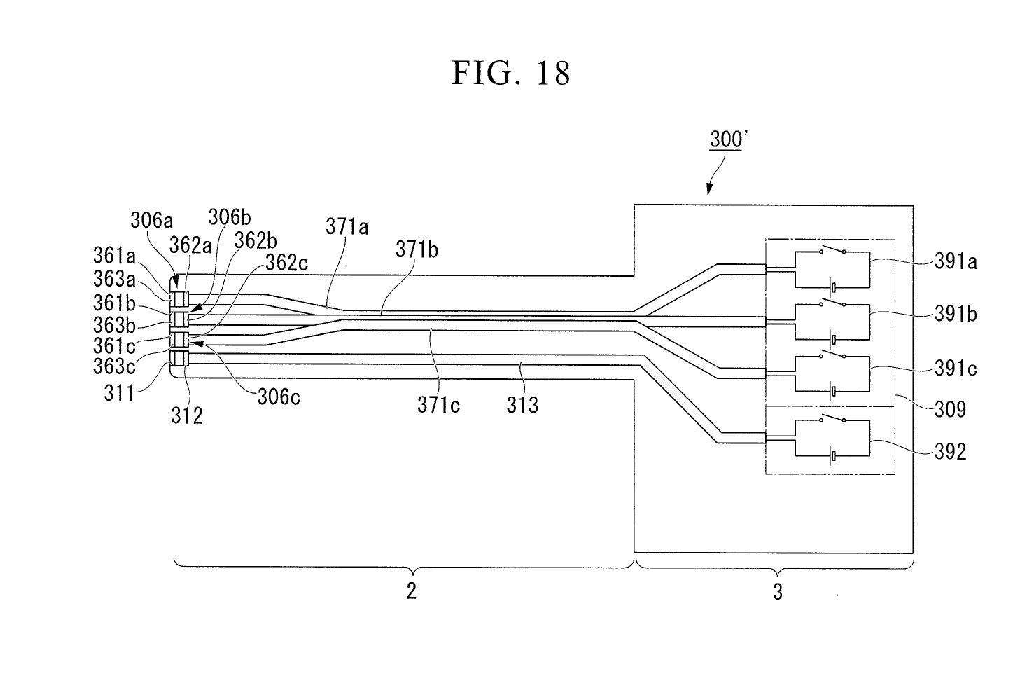

Further, as a modification of the fourth embodiment shown in FIGS. 17A to 18, an endoscope apparatus 300′ may further include a normal illumination window 311 provided in a distal surface 21A′ of a distal portion 21′, an emission member 312 such as an LED for the normal observation, a power source 392, and a power cable 313 which connects the emission member 312 and the power source 392. At the time of the normal observation, the light switch portion 309 turns off the power sources 391a, 391b, and 391c, and turns on the power source 392, in accordance with the control of the CPU 10. As a result, since light only from the on-state emission member 312 is projected on the subject, it is possible to observe the subject under light suitable for the normal observation.

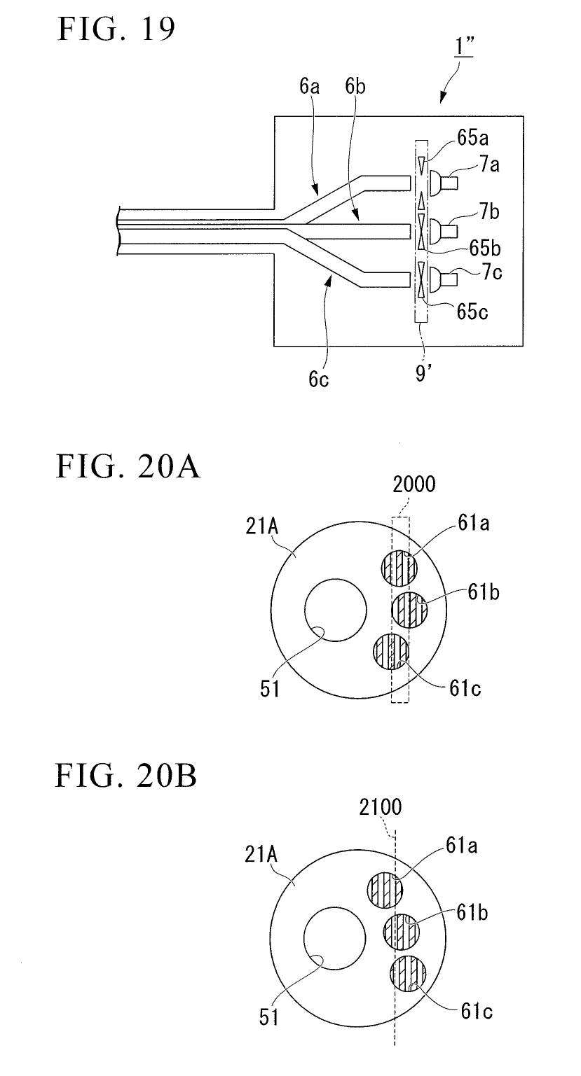

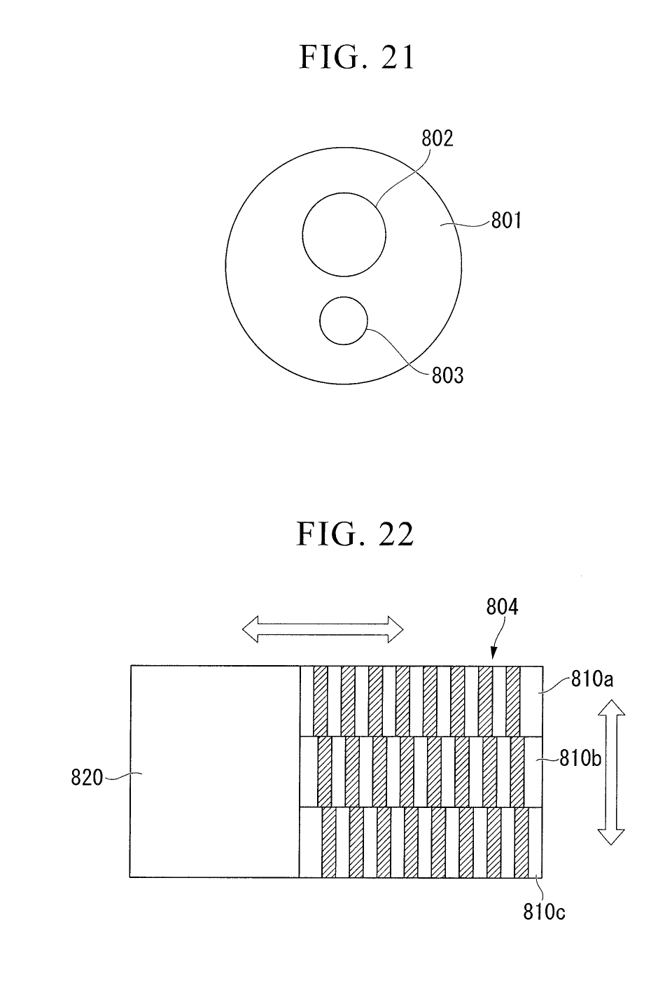

Further, in the above-described embodiments, the emission of light is switched by the light switch portion controlling on-off of the light devices. However, the present invention is not limited to this. For example, as another modification of the first embodiment shown in FIG. 19, an endoscope apparatus 1″ may further include open/close portions 65a, 65b, and 65c which are provided between the pattern windows and the light devices 7a, 7b, and 7c, respectively, and the emission of light may be switched by a light switch portion 9′ controlling opening/closing of the open/close portions 65a, 65b, and 65c. Since the emission of light is switched by opening/closing of the open/close portion 65a, 65b, and 65c, it is possible to keep the light devices 7a, 7b, and 7c on. This configuration is effective in the case where a light source such as a halogen lamp which requires long time or large power consumption for switching is used as the light device. In this case, although it is necessary to additionally provide an opening/closing mechanism for the open/close portions, this mechanism does not require an accurate positional control unlike a conventional mechanism for moving a pattern projection portion. Therefore, the apparatus does not become so complicated.

Further, in the above-described embodiments, endoscope apparatuses having three pattern projection units are described. However, the number of the pattern projection units is not limited to three, and it may be a counting number equal to or more than three. When it is assumed that the number of the pattern projection units is “n”, the pattern portions of the pattern projection units are disposed such that the line patterns of the pattern portions are shifted from each other by 1/n of the period P of the line pattern.

Further, in the above-described embodiments, the pattern windows are disposed such that the line connecting the centers of the pattern windows is parallel to the lines of the pattern portions. However, the present invention is not limited to this, and any arrangement of the pattern windows 61a, 61b, and 61c may be adopted as long as all of the pattern windows have an overlapped portion 2000 in the direction perpendicular to the line of the pattern portion as exemplified in FIGS. 20A and 20B. Further, a shape of the overlapped portion is not limited to a shape with width. A virtual line 2100 on the distal surface perpendicular to the line of the pattern portion which intersects each of the pattern windows may be adopted as the overlapped portion as shown in FIG. 20B.

What is claimed is:

1. An endoscope apparatus which is capable of performing measurement of a subject using a phase-shift method, the endoscope apparatus comprising:
a main body;
an insertion portion which is connected to the main body; and
a plurality of pattern projection units, each of the plurality of pattern projection units comprising:
a pattern window which is provided in a distal end of the insertion portion; and
a pattern portion which has a line pattern in which a plurality of lines are periodically disposed with a predetermined period, the lines being parallel to each other, wherein:
the pattern portions of the plurality of pattern projection units are disposed such that the line patterns of the pattern portions are shifted from each other by 1/n of the predetermined period of the line pattern, where the number of the plurality of pattern projection units assumed to be n (n≧3); and
the pattern windows of the plurality of pattern projection units are disposed such that all of the pattern windows have an overlapped portion in a direction parallel to the line of the pattern portion.

2. The endoscope apparatus according to claim 1, wherein each of the plurality of pattern projection units further comprises an emitting portion which emits light toward a corresponding one of the pattern windows.

3. The endoscope apparatus according to claim 2, further comprising light devices which are provided in the main body,
wherein each of the emitting portions is a light guide which connects a corresponding one of the light devices and a corresponding one of the pattern portions.

4. The endoscope apparatus according to claim 3, wherein:
the emitting portions are provided in the main body; and
each of the plurality of pattern projection units further comprises a coherent fiber which is provided in the insertion portion and connects a corresponding one of the pattern windows and a corresponding one of the pattern portions.

5. The endoscope apparatus according to claim 3, wherein the pattern portions are provided in a distal portion of the insertion portion.

6. The endoscope apparatus according to claim 5, wherein the light guides are coherent fibers.

7. The endoscope apparatus according to claim 3, further comprising a control portion which performs a control of switching the emission of light by controlling on-off of the light devices.

8. The endoscope apparatus according to claim 3, further comprising:
a plurality of open/close portions each of which is provided between a corresponding one of the light devices and a corresponding one of the pattern windows; and
a control portion which performs a control of switching the emission of light by controlling opening/closing of the plurality of open/close portions.

9. The endoscope apparatus according to claim 2, wherein the emitting portions are emission members.

10. The endoscope apparatus according to claim 9, further comprising a control portion which performs a control of switching the emission of light by controlling on-off of the emission members.

11. The endoscope apparatus according to claim 1, wherein each of the plurality of pattern projection units further comprises a projection optical system which is provided between a corresponding one of the pattern windows and a corresponding one of the pattern portions.

12. The endoscope apparatus according to claim 1, further comprising a control portion which performs a control so that light is emitted from all the emitting portions of the plurality of pattern projection units at the time of a normal observation in which the subject is observed without using the phase-shifting method.

13. The endoscope apparatus according to claim 1, further comprising a normal illumination window which is provided in the distal end of the insertion portion, and emits light to the outside at the time of a normal observation in which the subject is observed without using the phase-shifting method.

14. The endoscope apparatus according to claim 1, wherein:
the pattern windows of the plurality of pattern projection units have the same shape; and
the pattern windows are disposed such a line connecting the centers of the pattern windows is parallel to the lines of the pattern portions.

15. An endoscope apparatus which is capable of performing measurement of a subject using a phase-shift method, the endoscope apparatus comprising:
a main body;
an insertion portion which is connected to the main body; and
a plurality of pattern projection units, each of the plurality of pattern projection units comprising:
a pattern window which is provided in a distal end of the insertion portion; and
a pattern portion which has a line pattern in which a plurality of lines are periodically disposed with a predetermined period, the lines being parallel to each other, wherein:
the pattern portions of the plurality of pattern projection units are disposed such that the line patterns of the pattern portions are shifted from each other by 1/n of the predetermined period of the line pattern, where the number of the plurality of pattern projection units assumed to be n (n≧3); and
a virtual line on the distal end parallel to the line of the pattern portion intersects each of the pattern windows.

16. A measuring method of a subject using a phase-shifting method based on images obtained by sequentially projecting first, second, and third patterns on the subject, the first, second, and third patterns having a line pattern in which a plurality of lines parallel to each other are periodically disposed with a predetermined period and being disposed such that phases of the patterns are shifted from each other by a predetermined amount, the method comprising:
a first step of emitting light from a first light source to the subject via the first pattern and imaging a first subject image on which the first pattern is projected;
a second step of emitting light from a second light source different from the first light source to the subject via the second pattern and imaging a second subject image on which the second pattern is projected; and
a third step of emitting light from a third light source different from the first and second light sources to the subject via the third pattern and imaging a third subject image on which the third pattern is projected.
